(19) United States
(12) Patent Application Publication
(10) Pub. No.: US 2014/0112891 A1
(43) Pub. Date: Apr. 24, 2014

(54) AUTOLOGOUS HUMAN ADULT PLURIPOTENT VERY SMALL EMBRYONIC-LIKE (hVSEL) STEM CELL REGENERATION OF BONE AND CARTILAGE

(71) Applicants: Russell Taichman, Ann Arbor, MI (US); Paul H. Krebsbach, Ann Arbor, MI (US); Aaron Havens, Ann Arbor, MI (US); Anjali Mishra, Ann Arbor, MI (US); Denis Rodgerson, Malibu, CA (US); Jingcheng Wang, Ann Arbor, MI (US); Yusuke Shiozawa, Ann Arbor, MI (US); Younghun Jung, Ann Arbor, MI (US)

(72) Inventors: Russell Taichman, Ann Arbor, MI (US); Paul H. Krebsbach, Ann Arbor, MI (US); Aaron Havens, Ann Arbor, MI (US); Anjali Mishra, Ann Arbor, MI (US); Denis Rodgerson, Malibu, CA (US); Jingcheng Wang, Ann Arbor, MI (US); Yusuke Shiozawa, Ann Arbor, MI (US); Younghun Jung, Ann Arbor, MI (US)

(73) Assignees: NEOSTEM, INC., New York, NY (US); THE REGENTS OF THE UNIVERSITY OF MICHIGAN, Ann Arbor, MI (US)

(21) Appl. No.: 14/110,230

(22) PCT Filed: Apr. 9, 2012
(86) PCT No.: PCT/US2012/032807

§ 371 Date: Dec. 16, 2013

Related U.S. Application Data
(60) Provisional application No. 61/473,420, filed on Apr. 8, 2011.

(51) Int. Cl. A61K 35/28

(52) U.S. Cl. CPC A61K 35/28
USPC 424/93.7; 435/325

(57) ABSTRACT

The invention provides methods and compositions for the repair or regeneration of osteochondral tissue. The methods and compositions provide an effective amount of isolated differentiable human Very Small Embryonic Like Stem Cells (hVSELs) sufficient for regeneration or repair of an osteochondral tissue. The compositions can be administered directly to the tissue or administered systemically.

### CROSS-REFERENCE TO RELATED APPLICATIONS

This application claims priority to U.S. Application No. 61/473,420, filed Apr. 8, 2011, which is incorporated herein by reference in its entirety.

### STATEMENT OF GOVERNMENT FUNDING

This invention was made with government support under Grant Numbers AR056893 and DK082481 awarded by the National Institutes of Health. The government has certain rights in the invention.

### FIELD OF THE INVENTION

The present invention relates to compositions comprising very small embryonic-like (VSEL) stem cells and use of the compositions for treating bone and cartilage disorders in humans.

### BACKGROUND OF THE INVENTION

Adult human VSELs are a resident population of small pluripotent stem cells in the bone marrow that are involved in the normal turnover and regeneration of tissues. hVSELs are typically SSEA-4+/Oct-4+/CD133+/CXCR4+/Lin−/CD45−, express the pluripotency markers (Oct-4 and Nanog) and are capable of differentiation into cells from all three germ lineages including osteoblastic-like cells in vivo.

That their circulating levels greatly increase in response to injury and stress has been shown in animal models of myocardial infarction and in human myocardial infarction and stroke patients. VSELs also have been shown to be effective in repairing cardiac tissue in vivo. Furthermore, VSELs were found to rescue the immune system following radiation exposure. This suggests that stress promotes the release of VSELs from BM to allow these cells to traffic to the site of injury where they can promote recovery of the injured tissue by regenerating damaged tissue.

Unlike other adult stem cells (MSCs, iPSCs), human VSELs (hVSELs) can be used for a wide range of therapeutic applications because of their pluripotent characteristics.

Bone is a hard connective tissue consisting of cells embedded in a matrix of mineralized ground substance and collagen fibers. The fibers are impregnated with a form of calcium phosphate similar to hydroxyapatite as well as with substantial quantities of carbonate, citrate sodium, and magnesium; by weight, it is composed of 75% inorganic material and 25% organic material. Defects in the process of bone repair and regeneration are linked to the development of several human diseases and disorders, e.g. osteoporosis and osteogenesis imperfecta. Failure of the bone repair mechanism is also associated with significant complications in clinical orthopedic practice, for example, fibrous non-union following bone fracture, implant interface failures and large allograft failures. The techniques of bone reconstruction, such as is used to reconstruct defects occurring as a result of trauma, cancer surgery or errors in development, would also be improved by new methods to promote bone repair. Reconstructive methods currently employed, such as using autologous bone grafts, or bone grafts with attached soft tissue and blood vessels, are associated with significant drawbacks of both cost and difficulty. For example, harvesting a useful amount of autologous bone is not easily achieved, and even autologous grafts often become infected or suffer from resorption.

Parathyroid hormone (PTH) stimulates new osteoblasts in normal bone and at the site of bone wounds and fractures by increasing the pool of mesenchymal stem cells (MSC) available for growth plate expansion and fracture callus formation. Importantly, PTH can stimulate osteoblast progenitor cells during aging to maintain bone density and animal studies show that the increases induced in osteoblast formation are greater in older animals than younger ones. Both PTH 1-84 and PTH 1-34 have been shown to have a potent anabolic effect on bone in various animal models and humans over other established therapeutics. The efficacy of PTH is due to its ability to increase the pool of MSC in bone to stimulate new osteoblast formation to increase bone density, thus stem cell therapy may be more effective in stimulating new bone formation in a shorter time than PTH without the development of hypercalcemia.

The potential of very small embryonic-like (“VSEL”) stem cells to differentiate into osteoblasts and repair bone injuries is of great interest. Studies have been performed to test the ability of hVSELs to promote bone formation in a calvarial bone defect in Severe Combined Immunodeficiency (SCID) mice. These studies have shown that human VSELs (“hVSELs”) are able to differentiate to osteoblasts and regenerate bone tissue in vivo. Studies to test the ability of the autologous VSELs to promote bone remodeling in the human craniofacial skeleton that test the efficacy and safety of hVSELs in vivo in humans and provide the basis for the further development of these pluripotent stem cells to treat bone defects and loss are described.

### SUMMARY OF THE INVENTION

According to one aspect, the described invention provides a method for treating damage or an injury to osteochondral tissue comprising administering to the tissue an effective amount of a composition comprising autologous very small embryonic like stem cells (VSELs), wherein the VSELs differentiate to repair or regenerate the osteochondral tissue.

According to the invention, VSELs are contacted with human tissue in order to repair or regenerate osteochondral tissue, particularly bone and cartilage. Methods of mobilizing, collecting, and purifying VSELs have been described. In certain embodiments, VSELs thus obtained are administered directly to osteochondral tissue to be treated. In other embodiments, the VSELs are incorporated into a matrix, or scaffold, which may be biodegradable. In certain embodiments, the matrix is selected to elicit differentiation of the VSELs towards the desired tissue type. In certain embodiments, the VSELs are administered with an agent or growth factor that promotes differentiation towards the desired tissue type. In certain embodiments, the growth factor is a bone morphogenic protein (BMP).

In certain embodiments, method involves providing VSELs in a composition that is used to fill or coat a defect in an osteochondral tissue. The number of VSELs in the composition that is employed can be related to the volume or the surface area of the defect. In an embodiment of the invention, the composition comprises from about 20 to about 500,000 VSELs per mm³. In another embodiment, the composition comprises about 40 to about 4,000 VSELs per mm³. In an embodiment of the invention, the composition comprises from about 10 to about 100,000 VSELs per mm². In another embodiment, the composition comprises about 25 to about 500 VSELs per mm².

In certain embodiments, the VSELs are provided in a composition that comprises other nucleated cells. In certain such embodiments, the cells of the composition are at least about 50% VSELs. In other embodiments, at least about 70% of the cells of the composition are VSELs. In additional embodiments, at least about 90% or at least about 95% of the cells of the composition are VSELs.

The VSELs can be autologous, allogeneic, or engineered. In an embodiment of the invention, the method is used to treat or repair physical damage including, but not limited to, mending of broken bones, repair of cartilage tears, and the like. In other embodiments, the invention is used to regenerate tissue or to generate new tissue. Non-limiting examples include repair of restoration of cartilage damaged by arthritis or by general wear, and creation of bone in situ, for example to repair spinal or cranial defects. In certain embodiments, the method is used to promote adherence of artificial joints to skeletal bones. The method can be used to treat osteoarthritis, osteoporosis, or osteogenesis imprefecta.

The invention also provides a composition comprising an effective amount of VSELs sufficient for regeneration or repair of an osteochondral tissue. In certain embodiments, the stem cell composition comprises from about 20 to about 500,000 VSELs per mm³, or from about 40 to about 4,000 VSELs per mm³. In certain embodiments, the stem cell composition comprises from about 10 to about 100,000 VSELs per mm², or from about 15 to about 500 VSELs per mm². The stem cell composition may include other nucleated cells. In certain embodiments, the proportion of cells that are VSELs is at least about 50%, or at least about 70%, or at least about 90%, or at least about 95%.

According to the invention, the composition comprising VSELs may further comprise a matrix, or scaffold, which may be biodegradable. In certain embodiments, the matrix is selected to promote differentiation of the VSELs. In certain embodiments, the composition includes one or more agents or growth factors that promote differentiation, including, but not limited to bone morphogenic proteins (BMPs).

In one embodiment, the VSELs of the composition are differentiable to osteoblasts. In another embodiment, the VSELs of the composition are differentiable to chondrocytes.

### BRIEF DESCRIPTION OF THE FIGURES

FIG. 1 shows μCT and bone volume fraction resulting from human VSEL cell implantation into critical sized defects. μCT analysis of tissues generated by human VSELs in SCID mice. (A). Representative images of μCT bone formation following implantation of collagen based Gelfoam sponges or human VSELs (arrows). (B) Quantification of bone volume fraction obtained from n=10 implants from one of three donors. Enhanced bone formation compared to cellular control (human peripheral blood CD34+ mononuclear cells (donor 3) controls (carrier alone—Neg. control) was observed. Significant differences (*) from control at p<0.01.

FIG. 2 shows histologic examination of human VSEL cell ability to form bone in calvarial defects. Murine calvarial defects were filled with either a Gelfoam carrier containing either vehicle (A, E), human CD34+ cells (B, F) or Human VSEL cells (C, G—Donor 1, or D—Donor 2). At 3 months the calvarial defects were harvested, decalcified and hematoxylin and eosin staining performed. The data demonstrates that human VSEL cells generated bone tissues within the calvarial defects of human origin (See FIG. 4). Bar=100 microns.

FIG. 3 shows Masson's Trichrome stain showing hVSELs induce mineralization of calvarial defects. Analysis of the calvarial defect from mice treated with VSELs shows mineralization of the resulting bone tissue. Bone collagen stains blue, organic matrix components in bone that are not mineralized or “osteoid seams” stain red.

FIG. 4 shows immunohistochemistry for human HLA in bone formed in SCID mice following local transplantation of human VSEL cells. Bone/marrow interfaces were imaged following incubation with panhuman HLA antibody (D, E, F) or isotype matched controls (A, B, C) and counter-stained with DAPI to identify nuclei. Differential interference contrast (DIC) imaging of bone/marrow interfaces (A, D), immune florescence (IF) for the detection of human HLA (B, E) and superimposed images of DIC and immunohistochemistry. White arrows demonstrate osteoblasts. Bar=5 micron.

FIG. 5 shows endothelial cells in VSEL generated bone tissue are of human origin. Human specific antibody to the endothelial specific marker CD31, co-localized with human specific HLA markers, demonstrated human VSEL cells generated endothelial cells into a tubular structure in hVSEL implants (D, E, F) but not in negative control (A, B, C). Differential interference contrast (DIC) imaging of bone (A, D), immune florescence (IF) for the detection of human CD31 (B, E) and superimposed images of DIC and immunohistochemistry. White arrows demonstrate osteoblasts. Bar=5 micron.

FIG. 6 shows mineral content generated by hVSEL cells in immune deficient mice. (A) hVSEL cells were implanted into calvarial defects over a dose range of 2,000-500,000 cells/defect. At three months the tissue mineral content values within each defect were averaged for the animal groups. Positive controls included murine bone marrow stromal cells expressing BMP2, and negative controls consisted of the collagen carrier alone. (B) Averages of tissue mineral content formed within the calvarial defect by 2,000 hVSEL by donor. *P<0.05.

FIG. 7 shows histologic evaluations of tissues generated by hVSEL cells within calvarial defects. Representative slides were stained with H&E (top row) and Masson's trichrome (bottom row) in which collagen and bone appear blue. Positive controls included murine bone marrow stromal cells expressing BMP2 (not shown), negative controls (neg. control) consisted of only the collagen carrier alone. Histology presented at 20× (inserts) and 40× magnifications. Note the persistence of the collagen carrier matrix in the Neg. control group as well as the absence of an inflammatory cell infiltrate. In the 2,000 hVSEL groups demonstrate lamellar bone containing marrow spaces (arrows). Bar=100 microns.

FIG. 8 shows tissues generated by hVSEL cells are derived from human cells. Tissues formed by hVSEL cells within calvarial defects were immunostained with an fluorescent human specific pan-human leukocyte antigen (HLA) and merged with images of antinuclear stain (DAPI) and differential interference contrast (DIC) images. (A). Negative control is a longitudinal section of vessel in mice implanted with vehicle only demonstrating lack of human HLA staining (B-D) Longitudinal section of a vessel in mice injected with human VSEL cells demonstrating cytoplasmic human HLA staining Histologic images presented at 40×, Bar=100 microns.

FIG. 9 shows human osteocalcin present in murine serum. Circulating levels of intact human osteocalcin present in the serum (3 months) of animals implanted with nothing (negative control), carrier alone (sponge alone), murine bone marrow stromal cells expressing BMP, or 2,000-500,000 hVSEL cells/defect. Osteocalcin levels are presented as the mean and S.D. of all animals/implant group normalized against total serum protein. *P<0.05 compared to Negative control.

FIG. 10 shows human cells are found in the blood of experimental animals. Quantitative real-time PCR for human specific Alu was used to determine the presence of human cells within select murine tissues (spleen, femur, right lobe of the liver and whole blood). Pure mouse bone marrow served as a negative control and human bone marrow nucleated cells served as a positive control. Human DNA was not observed in the spleen, femur or liver of any of the animals implanted with 2,000 hVSEL cells. Human specific Alu was detected in the peripheral blood of the animals. *P<0.05 compared to Negative control.

FIG. 11 shows cartilage tissue generated by hVSEL cells are derived from human cells. Tissues formed by hVSEL cells were immunostained with an fluorescent human specific pan-human leukocyte antigen (HLA) and merged with images of collagen type II (Co II) and differential interference contrast (DIC) images.

### DETAILED DESCRIPTION OF THE INVENTION

The present invention provides a process for treating or regenerating osteochondral tissue in a subject. The process comprises obtaining an autologous population of very small embryonic-like stem (VSELs) cells and administering the cells to treat or regenerate osteochondral tissue or to generate new tissue. VSELs cells can be obtained from bone marrow or mobilized and collected from blood. Prior to administration, the VSELs are usually prepared by enrichment or purification, and may be incorporated into a support matrix. In certain embodiments, VSEL preparations of the invention further comprise one or more growth factors, including, but not limited to bone morphogenic factors or proteins (BMPs).

According to the invention, preparations of VSELs can be used to treat or regenerate connective tissue, including, bone and cartilage. The cartilage can be elastic cartilage, hyaline cartilage, or fibrocartilage. In certain embodiments, VSELs are used to repair articular cartilage (for example resulting from osteoarthritis). In another embodiments, VSELs are used to treat degenerative disc disease. The VSEL preparations of the invention are also useful for generating new bone and cartilage tissue, to be used, for example, in facial reconstruction.

The experimental results provided herein demonstrate that human VSEL cells are capable of generating osseous structures in a calvarial defect. The bone generated resulted in thick cortical structures surrounded by human osteoblasts which at the time of harvest (3 months) were mostly of a resting or lining cell phenotype. Abundant osteocytes were seen embedded in the cortical structures, and thick lamination of the cortical tissues were observed in Masons Trichrome Stain. To validate that the formed tissue was derived from the human VSEL cells, staining of the tissues with human specific pan-human leukocyte antigen antibody was performed. Only the tissues isolated from animals implanted with human VSEL cells demonstrated a marrow staining indicative of cells derived from the human donor cells. Histologic sections from purified and enriched hVSEL implants were also stained with antibodies specific for human osteocalcin (a specific mature osteoblastic marker).

Implantation of 200, 2,000, 10,000 VSEL cells per implant produced significant bone fill. Notably, larger numbers of VSEL cells per implant did not necessarily lead to faster or more complete regeneration. For example, more osseous tissue was generated in animals implanted with 2,000 cells/defect compared to defects implanted with 10,000 or 30,000 VSEL cells.

The term “administer” and its various grammatical forms as used herein means to give or to apply. The term “administering” as used herein includes in vivo administration, as well as administration directly to tissue ex vivo. Generally, compositions may be administered systemically either parenterally or topically in dosage unit formulations containing conventional nontoxic pharmaceutically acceptable carriers, adjuvants, and vehicles as desired, or may be locally administered by means such as, but not limited to, injection, implantation, grafting, topical application, or parenterally. The term “parenteral” or “parenterally” as used herein refers to introduction into the body by way of an injection (i.e., administration by injection), including, but not limited to, infusion techniques.

The term “autologous” as used herein refers to having originated from the same individual.

The term “cell differentiation” refers to the qualitative changes in morphology and physiology occurring in a cell as it develops from an unspecialized state into a mature or specialized cell type.

The term “damage” as used herein refers to physical harm caused to something in such way as to damage its value, usefulness or normal function.

The term “differentiable” as used herein refers to the ability to undergo cell differentiation.

The term “injury” refers to damage or harm caused to the structure or function of the body of a subject caused by an agent or force, which may be physical or chemical.

The term “isolated” is used herein to refer to material, such as, but not limited to, a cell, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 70%, 75%, 80%, 85%, 90%, 95%, 96%, 97%, 98%, or more than about 99% free of. The isolated material optionally comprises material not found with the material in its natural environment; or (2) if the material is in its natural environment, the material has been synthetically (non-naturally) altered by deliberate human intervention to a composition not native to a material found in that environment. The alteration to yield the synthetic material may be performed on the material within, or removed, from its natural state.

The term “multipotent” as used herein refers to the ability of a cell to become several different types of cells.

The term “osteoblasts” as used herein refers to cells that arise when osteoprogenitor cells or mesenchymal cells, which are located near all bony surfaces and within the bone marrow, differentiate under the influence of growth factors. Osteoblasts, which are responsible for bone matrix synthesis, secrete a collagen rich ground substance essential for later mineralization of hydroxyapatite and other crystals. The collagen strands form osteoids: spiral fibers of bone matrix. Osteoblasts cause calcium salts and phosphorus to precipitate from the blood, which bond with the newly formed osteoid to mineralize the bone tissue. Once osteoblasts become trapped in the matrix they secrete, they become osteocytes. From least to terminally differentiated, the osteocyte lineage is (i) Colony-forming unit-fibroblast (CFU-F); (ii) mesenchymal stem cell/marrow stromal cell (MSC); (3) osteoblast; (4) osteocyte.

The term “osteogenesis” refers to the formation of new bone from bone forming or osteocompetent cells.

Osteogenesis imperfecta (OI) refers to a group of inherited connective tissue diseases characterized by bone and soft connective tissue fragility (Byers & Steiner (1992) Annu Rev. Med. 43: 269 289; Prockop (1990) J. Biol. Chem. 265: 15349-15352). Males and females are affected equally, and the overall incidence is currently estimated to be 1 in 5,000-14,000 live births. Hearing loss, dentinogenesis imperfecta, respiratory insufficiency, severe scoliosis and emphysema are just some of the conditions that are associated with one or more types of OI.

The term osteoporosis refers to a heterogeneous group of disorders characterized by decreased bone mass and fractures. Clinically, osteoporosis is segregated into type I and type II. Type I osteoporosis occurs predominantly in middle aged women and is associated with estrogen loss at the menopause, while osteoporosis type II is associated with advancing age.

The term “pluripotent” as used herein refers to the ability of a cell to become every cell type in the body.

The term “stem cells” refers to undifferentiated cells having high proliferative potential with the ability to self-renew that can generate daughter cells that can undergo terminal differentiation into more than one distinct cell phenotype.

In some embodiments, the progressive composition of the described invention may be formulated with an excipient, carrier or vehicle including, but not limited to, a solvent. The terms “excipient,” “carrier,” or “vehicle” as used herein refers to carrier materials suitable for formulation and administration of the autologous stem cell product described herein. Carriers and vehicles useful herein include any such materials known in the art which are nontoxic and do not interact with other components. As used herein the phrase “pharmaceutically acceptable carrier” refers to any substantially non-toxic carrier useable for formulation and administration of the composition of the described invention in which the autologous stem cell product of the described invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “regeneration” as used herein refers to reproduction or reconstitution of a lost or injured part.

The term “repair” as used herein refers to restoration of diseased or damaged tissues naturally by healing processes or artificially.

The term “therapeutically effective” as used herein refers to the amount of the autologous stem cell product comprising human very small embryonic like stem cells (VSELs) that results in a therapeutic or beneficial effect following its administration to a subject. The therapeutic effect may be curing, minimizing, preventing or ameliorating a disease or disorder, or may have any other beneficial effect. The concentration of the substance is selected so as to exert its therapeutic effect, but low enough to avoid significant side effects within the scope and sound judgment of the physician. The effective amount of the autologous stem cell product may vary with the age and physical condition of the biological subject being treated, the severity of the condition, the duration of the treatment, the nature of concurrent therapy, the timing of the infusion, the specific compound, composition or other active ingredient employed, the particular carrier utilized, and like factors.

A skilled artisan may determine a pharmaceutically effective amount of the autologous stem cell product comprising human very small embryonic like stem cells (VSELs) by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED₅₀. The use of the term “individual” distinguishes the ED₅₀ based on the intensity of effect as used herein from the median effective dose, also abbreviated ED₅₀, determined from frequency of response data in a population. “Efficacy” as used herein refers to the property of the compositions of the described invention to achieve the desired response, and “maximum efficacy” refers to the maximum achievable effect. The amount of the autologous stem cell product that will be effective in the treatment of a particular disorder or condition will depend on the nature of the disorder or condition, and may be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001: THE PHYSICIAN'S DESK REFERENCE, Medical Economics Company, Inc., Oradell, N. J., 1995; and DRUG FACTS AND COMPARISONS, FACTS AND COMPARISONS, INC., St. Louis, Mo., 1993), each of which is incorporated by reference herein. The precise dose to be employed in the formulations of the described invention also will depend on the route of administration and the seriousness of the disease or disorder, and should be decided according to the judgment of the practitioner and each subject's circumstances.

As used herein the terms “treat” or “treating” are used interchangeably to include abrogating, substantially inhibiting, slowing or reversing the progression of a condition, substantially ameliorating clinical or aesthetical symptoms of a condition, substantially preventing the appearance of clinical or aesthetical symptoms of a condition, and protecting from harmful or annoying stimuli. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “very small embryonic-like stem cell” is also referred to herein as “VSEL stem cell” or “VSEL” and refers to certain stem cells that are pluripotent. In some embodiments, the VSEL stem cells (“VSELs”) are human VSELs and may be characterized as lin⁻, CD45⁻, and CD34⁺. In some embodiments, the VSELs are human VSELs and may be characterized as lin⁻, CD45⁻, and CD133⁺. In some embodiments, the VSELs are human VSELs and may be characterized as lin⁻, CD45⁻, and CXCR4⁺. In some embodiments, the VSELs are human VSELs and may be characterized as lin⁻, CD45⁻, CXCR4⁺, CD133⁺, and CD34⁺. In some embodiments, human VSELs express at least one of SSEA-4, Oct-4, Rex-1, and Nanog. VSELs may also be characterized as possessing large nuclei surrounded by a narrow rim of cytoplasm, and containing embryonic-type unorganized chromatin. In some embodiments, VSELs have high telomerase activity. In some embodiments, human VSELs and may be characterized as lin⁻, CD45⁻, CXCR4⁺, CD133⁺, Oct 4⁺, SSEA4⁺, and CD34⁺. In some embodiments, the human VSELs may be less primitive and may be characterized as lin⁻, CD45⁻, CXCR4⁺, CD133⁻, and CD34⁺. In some embodiments, the human VSELs may be enriched for pluripotent embryonic transcription factors, e.g., Oct-4, Sox2, and Nanog. In some embodiments, the human VSELs may have a diameter of 4-5 μm, 4-6 μm, 4-7 μm, 5-6 μm, 5-8 μm, 6-9 μm, or 7-10 μm. VSELs administered according to the invention can be collected and enriched or purified and used directly, or frozen for later use. Autologous or allogeneic VSELs can be administered according to the invention. Further, the VSELs may be engineered.

VSELs can be collected and purified by any method. WO/2011/069117 describes a method of isolation of stem cell populations from peripheral blood using sized-based separation. Fresh apheresed cells are lysed with 1×BD Pharm Lyse Buffer, in a ratio of approximately 1:10 (vol/vol) to remove red blood cells. After washing, cells are counted, and 2-2.5×10¹⁰ total nucleated cells are loaded onto the ELUTRA® Cell Separation System (CaridianBCT) at a concentration of 1×10⁸ cells/ml. Cells are then collected in 900 ml PBS+0.5% HSA media in each bag at different flow rates. Typically, six fractions are collected with a centrifugation speed of 2400 rpm. Finally, cells from all fractions are transferred into tubes and spun down at 600×g for 15 minutes. Size characteristics of the fractions are confirmed by evaluating SSC and FSC. As disclosed therein, Fraction 2 (50 mL/min) is highly enriched in VSELs and can be used to provide populations of VSELs for clinical applications. The procedure can be adapted to other equipment. The populations may be further purified by FACS.

In order to test the utility of human VSELs to form osteoblasts at sites of bone injury and to expedite the healing of bone injuries, VSELs were isolated from human volunteers using an apheresis and isolation procedure and then the regenerative properties of hVSELs to heal bone tested in SCID mice in a model of a calvarial defect.

hVSELs were applied to bone using Gelfoam™, an FDA approved scaffold (or matrix), to form osteoblasts at sites of bone injury and to expedite the healing of bone injuries in a cavarial bone defect in SCID mice. VSELs, obtained through apheresis from three different human donors and isolated by FACS, formed new bone when applied in the injured area as assessed by μCT scan to measure density. Histological analysis showed the hVSELs demonstrated osteogenesis, significant new bone formation, intact cortex-like structures, dense thickening of the trabeculae and bone marrow formation. Most importantly, new bone tissue was derived from the hVSEL since immunohistochemistry of the bone tissue using human specific HLA antibodies showed abundant human HLA labeling of marrow and of osteoblast-like cells adjacent to mineralized matrix. The studies described below provide the first evidence of the ability of hVSELs to differentiate to osteoblasts and generate new bone tissue in vivo and have the potential to repair bone injuries and treat osteoporosis. They also provide the foundation for studies in humans, testing for the first time, the ability of autologous human VSELs to promote bone remodeling in the human craniofacial skeleton and osteoporosis.

According to the invention, VSELs are administered in a therapeutically effective amount. In certain embodiments, the number of VSELs to be implanted depends on the volume of the composition that is administered. As exemplified, cell numbers ranging from 200 to 500,000 VSELs in a 3×3 mm collagen sponge were implanted in a 3 mm diameter calvarial defect (approximate volume=1.0-5.0 mm³), and mineralized tissue was produced. In certain embodiments, the number of VSELs administered in situ in a composition of the invention can be expressed in terms of cells per unit volume. In an embodiment of the invention, at least about 20 VSELs per mm³ are administered. In other embodiments, at least 40, at least 100, at least 200, at least 400, at least 1000, at least 2000, at least 5000, at least 10,000, at least 50,000, at least 100,000, or more VSELs per mm³ are administered. In certain embodiments of the invention, the range of VSELs per mm³ is from about 20 to about 500,000 or from about 40 to about 4000, or from about 20 to about 100 or from about 100 to about 400, or from about 400 to about 1000, or from about 1,000 to about 5,000, or from about 5,000 to about 50,000 VSELs per mm³.

In certain embodiments, the number of VSELs administered in situ in a composition of the invention can be expressed in terms of cells per unit area. In an embodiment of the invention, at least about 15 VSELs per mm² are administered. In other embodiments, at least 25, at least 100, at least 250, at least 500, at least 1000, at least 2000, at least 5000, at least 10,000, or at least 50,000 VSELs per mm² are administered. In certain embodiments, the range of VSELs per mm² is from about 10 to about 100,000, or from about 25 to about 500, or from about 10 to about 40, or from about 40 to about 100 or from about 100 to about 500, or from about 500 to about 2,500, or from about 2,500 to about 10,000, or from about 10,000 to about 100,000.

In certain embodiments of the invention, the number of VSELs administered to an osteochondral defect is from about 200 to about 1000. In certain embodiments, the number of VSELs administered is from about 1,000 to about 5,000. In certain embodiments, the number of VSELs administered is from about 5,000 to about 20,000.

The implantable compositions can comprise VSELs of varying purity. In one embodiment, the VSELs are at least 50% pure (i.e., represent at least 50% of nucleated cells). In other embodiments, the VSELS are at least 75%, at least 85%, at least 90%, or at least 95% pure. In another embodiment, the VSELs are from 50% to 80%, or from 80% to 90%, or from 90%-95%, or from 95%-99% pure.

In certain embodiments, the methods and compositions of the invention involve matrices suitable for osteogenic or chondrogenic growth. In bone, the extracellular matrix (ECM) consists of mainly of an organic phase known as osteoid, which constitutes approximately 20% of bone mass, and a mineral phase. The organic fraction of bone consists of over 90% type I collagen, other minor collagens such as types III and V, and 5% non-collagenous proteins. The non-collagenous proteins in bone include osteocalcin, osteonectin, osteopontin, adhesion proteins such as fibronectin and vitronectin and proteoglycans such as versican, decorin and hyaluronan. The mineral phase of bone is composed of hydroxyapatite, a calcium phosphate compound. The bone matrix also sequesters growth factors, acting as a reservoir for soluble inductive signals such as bone morphogenic protein (BMP).

In an embodiment of the invention, a matrix is used comprising VSELs dispersed within or on its surface. The matrix optionally includes an adhesive to hold the cells in position on a recipient organ surface. In some embodiments, the matrix is a sprayable, spreadable, or layerable fibrin glue (or fibrin sealant), comprising fibrinogen and thrombin. Two examples are Tisseel and DuraSeal. Such glues or sealants may be modified to adapt their density and degradation characteristics.

In certain embodiments, the matrix is a structure composed of a polymer, which may be biodegradable. In certain embodiments, the matrix is a polymer film, which may be free standing or coated on a support. In one embodiment, the polymer is poly(D,L-lactic-co-glycolic acid) (PLGA). In certain embodiments, the lactic acid-glycolic acid ration is 50:50, 65:35, or 75:25. In another embodiment, the polymer is polylactide (PLA). Other useful polymers include, without limit, chitosan, chitin, hyaluronan, heparin, heparin sulfate, chondroitin sulfate, keratan sulfate, and glycosaminoglycan.

ECM proteins are useful as scaffolds for bone defect healing and implant integration, and include collagen (such as Gelfoam™), fibrin, decellularized matrix, and bone sialoprotein. Artificial matrices can be functionalized with such proteins, for example by coating or tethering. Useful forms of ECM implants include crosslinked membranes, sponges, gels, demineralized bone particles or cut pieces of small intestinal submucosa. Some non-limiting examples are collagen, fibrin, DCM, and RGD peptides. RGD is a peptide sequence found in many ECM molecules including fibronectin, vitronectin, bone sialoprotein and osteopontin, and can bind to multiple integrins such αvβ3, αvβ1, α8β1, αvβ8, αvβ6, αvβ5 and αIIbβ3. (See, e.g., Shekeran et al., 201, J Biomed Mater Res A. 96(1): 261-72. Some clinically available bone graft materials are synthetic silicate-substituted porous hydroxyapatite (Actifuse ABX), synthetic alpha-TCP (Biobase), synthetic beta-TCP (Vitoss), synthetic beta-TCP (Chronos), processed human cancellous allograft (Tutoplast) and processed bovine hydroxyapatite ceramic (Cerabone).

In certain embodiments, the methods and compositions will further include osteogenic or chondrogenic growth factors. Such factors include, without limitation, BMP-2 (BMB-2a), BMP-3 (osteogenin), BMP-4 (BMP-2B), BMP-5, BMP-6 (Vgr-1), BMP-7 (OP-1), BMP-8 (OP-2), BMP-9 (GDF-2), BMP-10, BMP-11, BMP-12 (GDF-11, CDMP-3), BMP-13 (GDF-6, CDMP-2), BMP-14 (GDF-5, CDMP-1), BMP-15 (GDF-9B), BMP-16, BMP-17, BMP-18, and Vgr-2 (GDF-3). BMP-2, BMP-3, BMP-4, BMP-5, BMP-6 and BMP-7, are disclosed in U.S. Pat. Nos. 5,108,922; 5,013,649; 5,116,738; 5,106,748; 5,187,076; and 5,141,905. BMP-8 is disclosed in PCT publication WO91/18098, and BMP-9 is disclosed in PCT publication WO93/00432. BMP-10 is disclosed in U.S. Pat. No. 5,637,480, and BMP-11 is disclosed in U.S. Pat. No. 5,639,638. BMP-12 and BMP-13 are disclosed in U.S. Pat. No. 5,658,882. BMP-15 is disclosed in U.S. Pat. No. 5,635,372 and BMP-16 is disclosed in U.S. Pat. No. 5,965,403. BMP-17 and BMP-18 are disclosed in U.S. Pat. No. 6,027,917. Additional factors include FGF-1, FGF-2, IFG-1, IGF-2, TGF-β1, TGF-β2, TGF-β3, and VEGF. In certain embodiments, one or more bisphonates (e.g., etidronate, clodronate, alendronate, pamidronate, risdronate, zoledronate) is included.

In certain embodiments, the VSEL compositions of the invention comprise one or more blood components, including, but not limited to, erythrocytes, leukocytes, monocytes, platelets, or platelet rich plasma.

The structure of the matrix can be modified to whatever shape and dimension is best suited to put VSELs in contact with or proximate to the injury or defect. The matrix comprising VSELs can be in the form of a patch, wrap, or conduit, and may be configured to fit the contours and dimensions of the injury or defect. In certain embodiments, the VSELs seeded matrix is positioned such that the majority of VSELs are in direct contact with the defect. In other embodiments, the VSELs are placed in proximity to the injury or defect.

The invention provides methods of treating a human or other mammal having a bone or cartilage defect by administering a composition comprising VSELs. Tissue defects include, without limitation, congenital defects, results or symptoms of disease or trauma, or resulting from surgical or other medical procedures.

Bone fractures include injuries in which a bone is cracked, broken, or chipped. Bone healing occurs naturally in most subjects. Fracture is normally followed by bleeding and clotting, production of collagen by fibroblasts and mineralization of the collagen matrix. Over time, the resulting immature bone undergoes a remodeling to produce mature lamellar bone. However, failures of fracture repair (nonunions) occur in 10% of all fractures. The VSEL compositions and methods facilitate normal bone healing and remodeling and reduce the occurrence of nonunions.

The VSEL compositions and methods are also used to promote formation of new bone at a desired location. Nonlimiting examples include implanted appliances and prostheses. According to the invention, an implanted appliance or prosthesis is formed from or coated with a material that is impregnated with VSELs. “Impregnated” means the material comprises VSELs on its surface and/or within.

Thus, the disclosed VSEL compositions are useful for repair, regeneration, and growth of bone. Nonlimiting applications include joint replacement surgery, not limited to hip replacement, knee replacement, shoulder replacement, and ankle replacement, bone fusion, including spinal fusion, joint fusion, including fusion of bones of the wrist, fingers, toes, and spine, cranioplasty, dental bone grafts and implant placement, and rebuilding or replacing bone lost to disease such as cancer.

VSEL compositions of the invention are useful to treat osteoporosis. In certain embodiments of the invention, VSELs are administered systemically (e.g. intraveneously) in order for the VSELs cells to access the entire skeletal structure. For example, VSELs express CXCR4 and respond to a CXCL12 (SDF-1) gradient, and CXCL12 and other chemoattractants are secreted by bone marrow stromal cells. In certain embodiments of the invention, at least 5×10³, or at least 10⁴, or at least 5×10⁴, or at least 10⁵ or at least 5×10⁵, or at least 10⁶ VSELs are administered. In certain embodiments the range of VSELs administered is from about 10³ to about 10⁴, or from about 10⁴ to about 10⁵, or from about 10⁵ to about 10⁶.

In an embodiment of the invention, an agent that promotes homing and/or adherence of VSELs to bone tissue is employed. One such agent comprises a first portion that binds to VSELs and a second portion that binds to bone tissue. Agents that bind to VSELs include, without limitation, antibodies specific for VSEL markers (e.g., CXCR4, CD133). Agents that bind to bone include, without limitation, bisphosphonates (e.g., alendronate), which have also been used to target proteins and MSCs to bone. In certain embodiments, the first portion may be specific for a marker expressed on VSELs and other cells. In such cases, it may be preferable to incubated the agent with purified VSELs prior to administration. In another embodiment of the invention, an agent that binds to bone tissue can be covalently linked to the VSEL. In such cases, the agent can be linked to any VSEL component prior to administration. In an embodiment of the invention, a subject with osteoporosis is treated with a VSEL mobilizing agent and an agent that promotes homing and/or adherence of VSELs to bone tissue.

Articular cartilage covers the ends of bones in diarthroidial joints in order to distribute the forces of locomotion to underlying bone structures while simultaneously providing nearly frictionless articulating interfaces. These properties are furnished by the extracellular matrix composed of collagen types II and other minor collagen components and a high content of the proteoglycan aggrecan. Low friction properties are the result of a special molecular composition of the articular surface and of the synovial fluid as well as exudation of interstitial fluid during loading onto the articular surface. Articular cartilage has a limited response to injury in the adult mainly due to a lack of vascularisation and the presence of a dense proteoglycan rich extracellular matrix.

The present invention provides a VSEL composition and method for use in repair, regeneration, reconstruction or bulking of cartilaginous tissue. Preferably, the composition adheres to the cartilage tissue in which it is introduced and supports VSEL differentiation and proliferation for repairing the cartilage. In one embodiment, the composition comprises a polymer composition which when mixed with VSELs and any other desirable components becomes non-liquid (e.g., gels) such that the composition is retained at and adheres to the site of introduction. The polymer can be a modified or natural polysaccharide, such as chitosan, chitin, hyaluronan, glycosaminoglycan, chondroitin sulfate, keratan sulfate, dermatan sulfate, heparin, or heparin sulfate. In some cases, the cartilage at the site of introduction is prepared by piercing, abrading, or drilling to provide a void or location for the VSEL composition and/or to facilitate engraftment of the VSELs. The methods and compositions are suitable for treating injuries to cartilage, including, without limitation, partial thickness (part of the way down to bone) or full-thickness (all the way down to bone) injuries and meniscus tears. Diseases involving degeneration of cartilage that can be treated include, without limitation, osteoarthritis, rheumatoid arthritis, psoriatic arthritis, lupus, gout, and Lyme disease.

In certain embodiments, allogeneic VSELs are used to treat damaged or injured to osteochondral tissue. Allogeneic VSELs are preferred for treating skeletal conditions that have genetic origins. For example, in an embodiment of the invention, allogeneic VSELs are administered to a subject to treat osteogenesis imperfecta. This disease is often characterized by too little type I collagen or a poor quality of type I collagen due to a mutation in one of the type I collagen genes. Alternatively, a subject's own VSELs, engineered to express type I collagen upon differentiation, can be employed.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a,” “and,” and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### VSELs can Differentiate In Vivo to Osteoblastic-Like Cells

Initial studies focused on establishing the ability of murine VSELs to differentiate into osteoblasts, the critical cell type involved in bone formation. Dr. Taichman and his group had recently described an in vivo assay that could be used to identify cells with stem-like activities. They had found that cells with MSC-like activity were present in murine BM at low density which were resistant in vivo to 5-fluorouracil (5-FU) and expressed CXCR4 [46,47]. These cells were characterized by FACS and subsequently found to be Lin−Sca-1+CD45-cells with the same properties as VSELs [3-5].

To evaluate the potential of murine VSELs to undergo multi-lineage differentiation, intra-marrow transplantation studies were performed. First, MSCs were harvested from Col2.3Δ TK mice, expanded in culture, and implanted into SCID mice to generate a recipient site in which thymidine kinase tissues would be established. The rationale for using this strategy was to be able to ablate osteoblast numbers in the resulting marrow of the implant to clear space for injection of the GFP marked cells to undergo lineage progression. As controls, mRNA from freshly isolated VSELs was evaluated for the expression of the osteoblast specific marker Runx-2 and the adipocyte marker PPARγ. Compared with the osteoblastic cell line MC3T3-E and bone marrow stromal cells treated with 1×10⁻⁶ M dexamethasone, VSELs expressed little if any mRNA for Runx-2 and PPARγ [47]. At 1.5 months, the ossicles were surgically exposed and injected with VSELs isolated from GFP mice. After an additional 1.5 months, the implants were harvested and differentiation of the injected GFP cells was determined by cell surface markers and confocal microscopy. Co-localization of GFP expressing cells with an antibody to the osteoblast specific marker Runx-2 and the adipocyte marker PPARγ was performed. Approximately half of the Runx-2 expressing cells also expressed GFP. Similarly, nearly half of the PPARγ-expressing cells were marked with GFP. These data indicate that lineage progression down at least two lineages from the VSELs had occurred in vivo. Most importantly, VSELs were able to differentiate into osteoblastic-like cells in vivo.

### Example 2

### Human VSELs can Regenerate Bone in a Calvaria Bone Defect Model

These initial studies provided the foundation for studies to test the efficacy of human VSELs to form bone in the calvaria bone defect model. For these studies, NeoStem isolated VSELs for healthy human volunteers. The individuals were treated over the course of 3 days with G-CSF (480 ug/day) and a 200 ml blood sample was drawn and subjected to apheresis and FACS to isolated VSELs. VSELs at ˜70% or ˜90% purity were isolated along with CD34+ cells (VSELs are CD34−) which were used as negative cellular controls, and loaded into collagen Gelfoam™ sterile sponges as an inert carrier.

Five-week-old female SCID mice (N:NIH-bg-nu-xid; Charles River Labs) were divided randomly into groups consisting of 10 each. To generate the calvarial defect, a linear scalp incision was made and bilateral full-thickness flaps were elevated. The periosteum overlying the calvarial bone was resected and a trephine bur with water spray was used to create a 5-mm craniotomy defect. After removing the calvarial disks, the VSEL cells or control (negative—Gelfoam only or CD34+ cells) were placed into the defects. The incisions were closed with 4-0 Chromic Gut suture (Ethicon/Johnson & Johnson, NJ), and the mice recovered. All mice were sacrificed 3 months post-surgery. The calvaria were harvested, immediately fixed in 10% neutral buffered formalin for 48 h, and then scanned for μCT analysis and subsequently decalcified with a 10% EDTA solution for 2-3 weeks, dehydrated with gradient alcohols, and embedded in paraffin for histology.

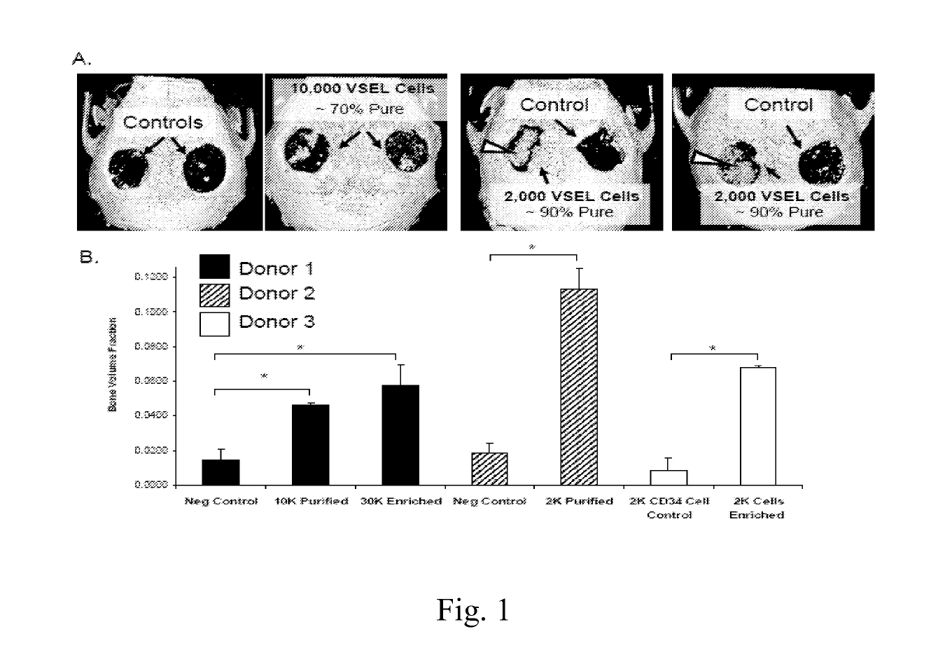

Specimens were scanned at 8.93 μm voxel resolution on an EVS Corp., μCT scanner (London, Ontario, Canada), with a total of 667 slices per scan. GEMS Micro View® software was used to make a 3-D reconstruction from the set of scans. A fixed threshold (1,000) was used to extract the mineralized bone phase, bone volume fraction (BVF), bone mineral density (BMD) and trabecular number (Th.N.). Representative images and quantification for bone volume fraction is provided in FIG. 1A, B. The data demonstrate that animals implanted with carrier (control) or CD34+ cells alone did not generate any bone tissues (FIG. 1A, first panel and panels 3 and 4, right side defect and 1B). Implantation of 10,000 purified VSEL cells from healthy donor 1 both stimulated bone formation (FIG. 1A, second panel from the left). There appeared to be a dose dependency of the efficacy of VSELs from donor 1 to generate bone, although this difference did not reach statistical levels (FIG. 1B). Bone generated by VSELs from volunteers 2 and 3 (FIG. 1A, third and fourth panels from the left) provided even greater bone fill compared to VSELs from donor 1, even when fewer cells were used suggesting potential individual differences in VSELs ability to promote bone formation.

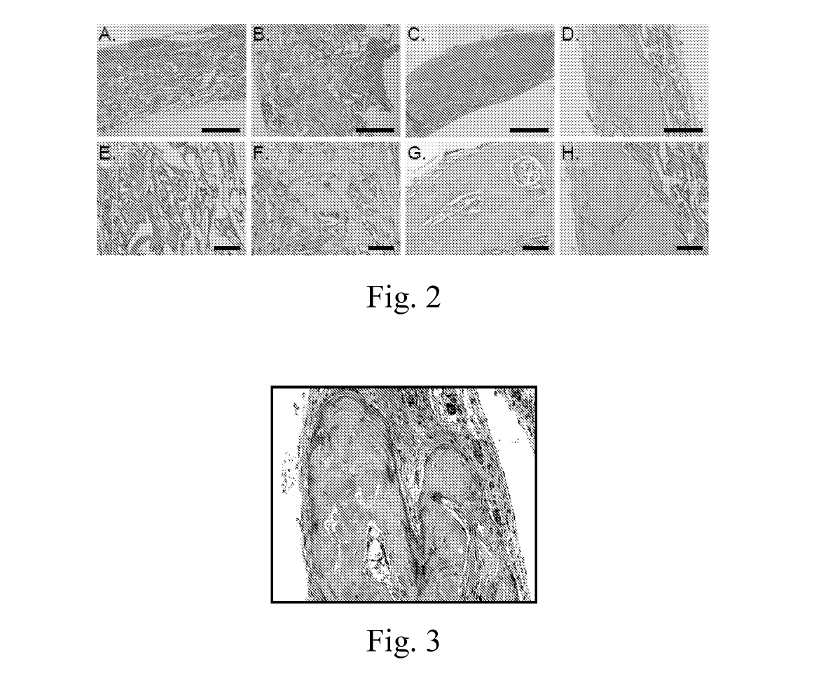

Histologic examination of the control Gelfoam sponges transplanted without cells resulted in only low levels of neutrophils and connective tissue in growth (FIG. 2 A,E). Implantation of 2×10⁴ CD34+ cells also failed to demonstrate specific tissue regeneration and consisted of loosely organized connective tissue and residual collagen scaffold material (FIG. 2B, F). Implantation of 2,000 human VSELs from all three donors produced small focal areas of hypertrophic cells that resembled bone-like tissues (FIG. 2C, D). At higher magnification the VSEL treated groups demonstrated significant new bone formation, adipocytes and the recruitment of hematopoietic marrow. The extent of osteogenesis appeared to be inversely dependent on the number of cells seeded, with the 2,000 VSEL cells demonstrating more intact cortex-like structure, denser thickness of trabeculae and more bone marrow than those of 10,000 cells again consistent with complex interactions occurring between and amongst stem cells and potentially support or accessory cells. Transplants without cells resulted in only low levels of neutrophils and connective tissue in growth (FIG. 2A, E). Implantation of 2×10⁴ CD34+ cells also failed to demonstrate specific tissue regeneration and consisted of loosely organized connective tissue and residual collagen scaffold material (FIG. 2B, F). Implantation of 2,000 human VSELs from all three donors produced small focal areas of hypertrophic cells that resembled bone-like tissues (FIG. 2C, D). At higher magnification the VSEL treated groups demonstrated significant new bone formation, adipocytes and the recruitment of hematopoietic marrow.

Evidence that the VSELs caused mineralization of the bone is shown in Masson's Trichrome staining (FIG. 3). The stain shows collagen as blue and the VSEL treatment greatly increasing collagen deposits in the calvarial defect compared to regions that are not mineralized (red stain). In fact, most of the defect is filled by newly formed bone tissue.

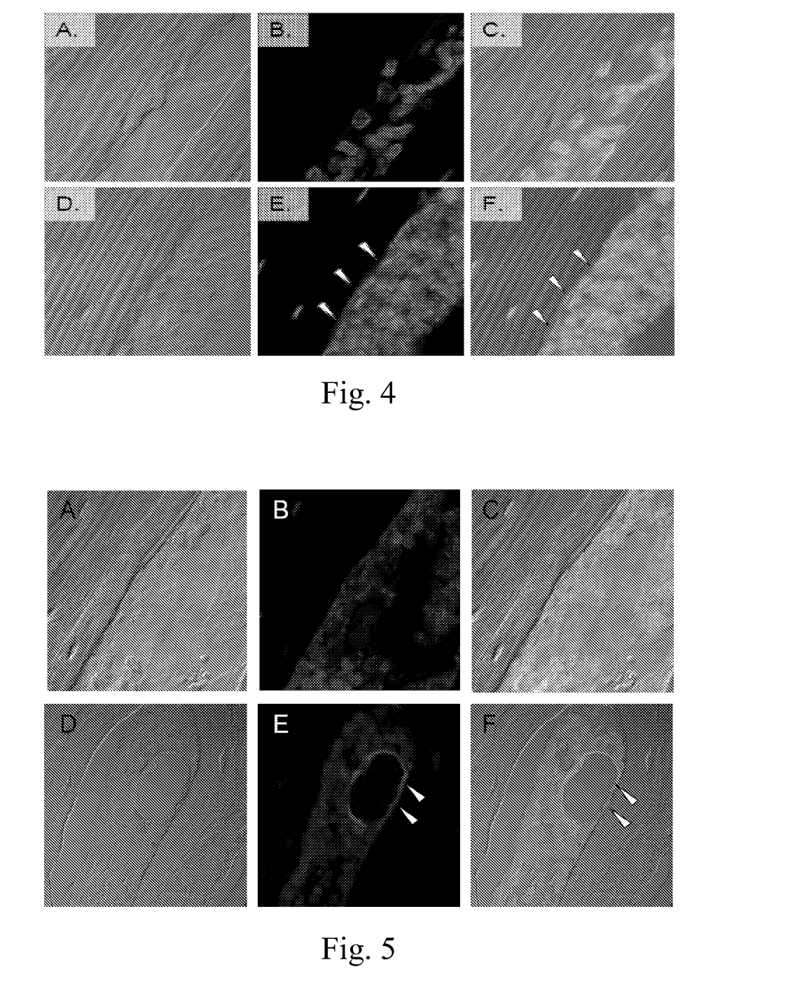

To determine whether the bone tissues were derived from human or murine sources, sections were stained with antibodies specific for human leukocyte antigen (HLA), the major histocompatibility complex (MHC) in humans or an isotype control (FIG. 4).

Immunohistochemistry for human HLA identified abundant marrow staining Osteoblast-like cells adjacent to mineralized matrix were particularly strongly stained for human HLA, whereas adjacent sections containing human cells failed to stain in the presence of IgG control stain. These data demonstrate that that the transplanted human VSEL cells contributed to generating new bone tissues.

Endothelial cells are critical for bone formation. We therefore evaluated whether endothelial cells were formed within the osseous tissue. Using human specific antibody to the endothelial specific marker CD31, co-localized with human specific HLA markers demonstrated that in fact the human endothelial cells into a tubular structure occurred in hVSEL implants but not in negative control (FIG. 5).

The results establish that VSELs can be isolated from human volunteers in sufficient quantities (approximately 17 million cells per 200 ml TNC apheresis) to be able to use for regenerative medicine. Studies establishing the procedures to isolate VSELs from human blood have been published [28] and are incorporated herein by reference.

Furthermore, the fact that bone was able to be formed with 2000 human VSELs in the mouse model (FIG. 1) indicates that expansion of the cells once isolated, induction of differentiation, or any other manipulation of the cells is unnecessary. In fact, the studies on bone regeneration are all done with freshly isolated human VSELs. We have shown that VSELs can differentiate in vivo into osteoblastic-like cells as indicated by their expression of Runx-2 when implanted into host bone marrow. (Taichman, 2010).

Most importantly, we have shown that hVSELs can generate new bone in injured bone (FIGS. 3-6). These are the first in vivo studies of hVSEL differentiation and the first evidence of their regenerative capabilities. The results support their in use in regenerative medicine, in particular in bone repair.

Finally, our studies provide information on how best to administer hVSELs to regenerate bone. First, Gelfoam appears as a useful scaffold to apply the cells to bone wounds. Gelfoam is FDA approved for use in the treatment of wounds and internal organ injury and has been used extensively in human studies. Therefore, Gelfoam should be usable as a scaffold to apply hVSEL to human bone to treat injuries and bone loss.

### Example 3

### Human VSELs can Regenerate Bone in a Calvaria Bone Defect Model

Healthy Caucasian males and females were recruited as VSEL cell donors and screened for known diseases, use of drugs and tobacco and obesity. Two days prior apheresis, each donor received daily subcutaneous injections of G-CSF (Granulocyte-Colony Stimulating Factor (Neupogen®, Amgen Inc., Thousand Oaks, Calif.)) (480 μg/day) to facilitate mobilization of VSEL cells in the bone marrow that are subsequently released into the blood stream. Apheresis was conducted by a certified staff technician over the course of 2 to 3 hours. Subsequently the human VSEL cells were enriched by Elutriation (CaridianBCT), followed by CD34/CD 133 Microbeads (Miltenyi Biotec) positive selection, then viable Lin−CD45−CD34+CD133+VSEL cells were flow sorted using Moflo XDP high speed cell sorter (Beckman Coulter). High purified VSEL cells were finally frozen in PBS-5% HSA and shipped by overnight courier to the University of Michigan without any demographic information.

Five-week-old female SCID mice (N:NIH-bg-nu-xid; Charles River Laboratories, Raleigh, N.C.) were divided randomly into n=5 groups consisting of n=10-13 animals. The animals were anesthetized by isoflurane inhalation and a linear scalp incision was made from the nasal bone to the occiput, and full-thickness flaps were elevated. A trephine was used to create a 3-mm craniotomy defect centered in each of the parietal bones while irrigated continuously with Hanks' balanced salt solution. The calvarial disks were removed to avoid injury of the underlying dura and brain tissues. After hemostasis was established, scaffolds (Gelfoam™, Pharmacia & Upjohn, Kalamazoo, Mich.) previously loaded with either vehicle or hVSEL cells were placed into the defects with care taken so that the scaffolds filled the entire defect and attached the bone edges at the surgical periphery. The incisions were closed with 4-0 Chromic Gut suture (Ethicon/Johnson & Johnson, Somerville, N.J.), and the mice recovered from anesthesia on a heating pad. All mice were sacrificed 16 weeks after the implantation. At sacrifice, intracardiac puncture and aspiration was performed under anesthesia to collect serum.

Bone marrow was isolated by flushing the femurs, tibia and humeri of C57BL/6 mice (Jackson Laboratory) with DMEN+10% FBS (Invitrogen, Grand Island, N.Y.). Plastic adherence at 37° C. was performed in modified Dexter's medium (IMDM medium, 10% FBS, 10% equine serum, 1 μM hydrocortisone, penicillin/streptomycin (Life Technologies, Grand Island N.Y.)). Following overnight adherence, the non-adherent cells were removed and fresh medium was replaced. The cultures were expanded by trypsinization twice over the course of three weeks generating first and second passage cells (P₁ or P₂). BMSCs from P₁ or P₂ at 80-90% confluence were transduced with AdCMVBMP-7 ex vivo 24 hours prior to transplantation at a multiplicity of infection (MOI) of 500. The AdCMVBMP-7 was constructed by Cre-lox recombination as previously described (Franchesi et al., 2000, J. Cell. Biochem 78:476-86) and generated by Vector Core at the University of Michigan.

Five groups of mice were established to evaluate the ability of human VSEL cells to regenerate the craniofacial defect. The first group served as a negative control in which only the vehicle and Gelfoam™ were placed into the defect. The second group consisted of P₁ or P₂ murine bone marrow stromal cells infected with an AdCMVBMP-7 designed to express huBMP-7 to serve as a positive control. Test groups consisted of 20, 200 or 2000 VSEL cells in Gelfoam™ isolated from three different individuals. The cell doses were arrived at by estimating the (i) frequency of these marrow human MSCs reported to be present in bone marrow (ranging from 1/10,000 to 1/100,000 bone marrow mononuclear cells and observations that ˜2×10⁶ human marrow adherent cells are required to heal a 3 mm cranial defect in mice. The incorporation of a 2000 VSEL cell dose was to ensure our ability to observe a VSEL cell response assuming that only 10% of the transplanted cells were able to participate in wound repair.

Micro Computed Tomography (μCT).

Calvaria were harvested and immediately fixed in 10% neutral buffered formalin for 48 h. The bones specimens were then scanned at 8.93 μm voxel resolution on an EVS Corp., microCT scanner (London, Ontario, Canada), with a total of 667 slices per scan. GEMS MicroView® software was used to make a 3-D reconstructions from the set . Each defect was individual assessed for the region of interest (ROI) and bone analysis was conducted with a fixed threshold (600) used to extract the mineralized bone phase, bone volume fraction (BVF), bone mineral density (BMD) and trabecular number (Th.N.) was calculated.

Histologic Examination.

After μCT analysis, the bones were decalcified in 10% EDTA (pH 7.4) for 10 days and embedded in paraffin. Longitudinal sections of the calvaria were cut and stained with hematoxylin and eosin (H&E) or using Masson's trichrome staining and analyzed by light microscope. In some cases, the slides were stained with an antibody to human HLA antigens (Anti-HLA-ABC antibody (BioLegend, San Diego, Calif.)) or an IgG control (Sigma) in conjunction with a HRPAEC staining system kit following the manufactures protocols (R&D Systems) to identify the human cells.

Serum Osteocalcin Levels.

Human osteocalcin levels were determined using a sandwich Mid-Tact Osteocalcin EIA (Biomedical Technologies, Stoughton, Mass.) and using human recombinant osteocalcin as a standard (Biomedical Technologies). This sandwich EIA is highly specific for both intact human osteocalcin and the major (1-43) fragment. In order to normalize the resulting osteocalcin levels in the serum, total protein was determined using the RC-DC Protein Assay Kit (BioRad Laboratories) against a bovine serum albumin standard.

Real Time PCR Evaluation for Human VESL.

DNA isolation kits were used to prepare genomic DNA from the designated tissues (DNeasy Blood and Tissue Kit (Cat. no. 69506); Qiagen, Inc., Valencia, Calif.). All sample concentrations were standardized in each reaction to exclude false-positive results. Real-time polymerase chain reactions were performed using 15.0 μl of TaqMan PCR Master Mix (Applied Biosystems, Foster City, Calif.) with 100 nM of the human Alu TaqMan probes (Forward -5′-CATGGTGAAACCCCGTCTCTA-3′, Reverse-5′-GCCTCAGCCTCCCGAGTAG-3′, TaqMan probe-5′-FAM-ATTAGCCGGGCGTGGTGGCG-TAMRA-3′); (Applied Biosystems) and 1 μg of the isolated tissue DNA in a total volume of 30 μl. The thermal conditions were 50° C. for 2 minutes, 95° C. for 10 minutes followed by 40 cycles of 95° C. for 15 seconds and 60° C. for 1 minute. The level of expression was detected as an increase in fluorescence using a sequence detection system (ABI PRISM 7700; Applied Biosystems). The DNA levels were expressed as relative copies (% control) normalized against murine β-actin (Cat. no. 4331182; Applied Biosystems), and a standard curve constructed from serial dilutions of a purified Luc/Alu cDNA fragment was cloned by classic PCR. Numerical data were determined against a standard curve established using mouse bone marrow containing log-fold dilutions of human VSEL. Positive and negative controls included tissues obtained non-VSEL injected mice or DNA derived directly from VSEL.

Statistical Analyses.

Numerical data are expressed as mean±standard deviation. Statistical analysis was performed by Kruskal-Wallis one-way analysis of variance using the GraphPad Instat statistical program (GraphPad Software, San Diego, Calif.). Specific differences were measured by Mann-Whitney U test. Level of significance set at P<0.05.

Evaluation of Bone Formation by Micro Computed Tomography.

Human VSEL were evaluated for their ability to form bone in murine calvarial defects. VSEL cell were isolated following G-CSF mobilization of normal healthy donors and placed into calvarial defects generated in the left parietal bones measuring 3 mm in diameter. Transplanted cells (ranging from 2,000-500,000 cells) were delivered to the defects in 3×3 mm Collagraft™ collagen sponges. Negative cellular controls consisted of the sponge alone. Positive controls incorporated murine bone marrow stromal cells engineered to over express hBMP7. After 3 months all the specimens were evaluated by μCT. The data demonstrated that animals implanted with carrier (negative control) alone did not generate any bone tissues compared to the mineralized tissue formation in the positive control. Implantation of 2,000-500,000 VSEL cells from all three of the tested donors all stimulated bone formation. Examination of the mineralization demonstrated incomplete closure of all of the defects. Nevertheless, robust bone formation was observed in samples generated from the 2,000 and 10,000 cells/implant groups. Interestingly, bone formed in animals treated with 30,000 cells/implant did not produce significantly more osseous tissue than those treated with 2,000 cells/implant, while 500,000 cells/implant produced greater boney structures than did the 30,000 and 2,000 cells/implant groups.

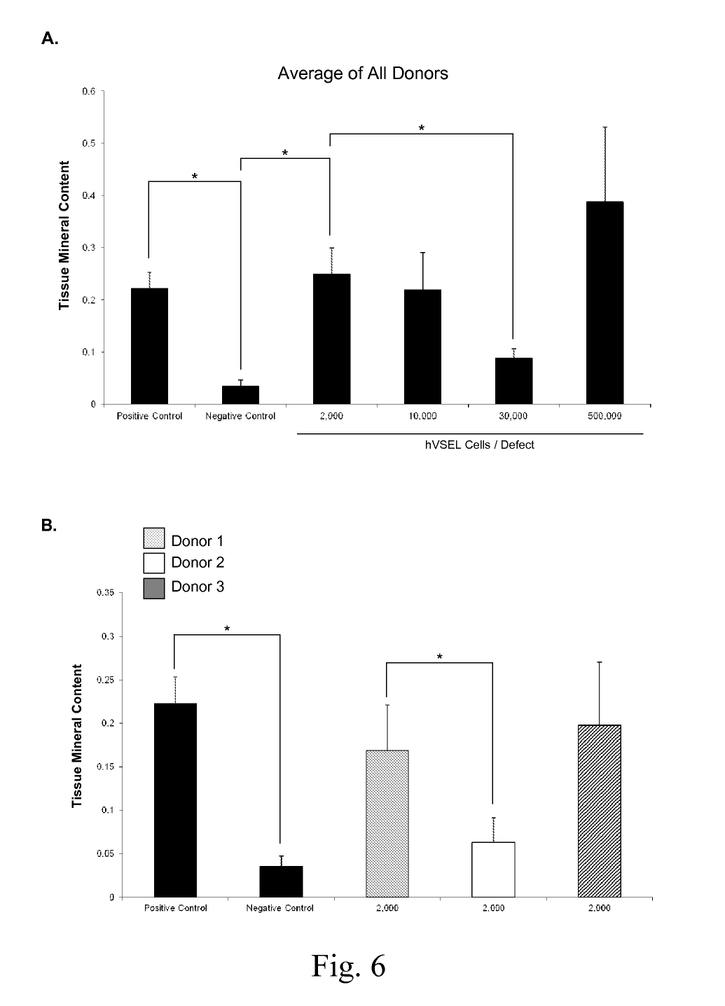

Using the GEMS MicroView software, each sample was examined for mineral content. The efficacy of cells to produce mineralized matrix appeared to be inversely related to the dose of cells utilized, although this difference did not reach statistical levels (FIG. 6A). For example, defects implanted with 2,000 human VSELs produced more mineralized tissue than those implanted with 10,000 or 30,000 cells. Interestingly, implants with 30,000 hVSELs produced the lowest mineralized tissue. When calvarial defects were implanted with 500,000 cells, mineralized tissue formation was maximized although not to a statistically significant amount.

As previously described, hVSEL isolation was collected from three separate donors. To assess any differences in efficacy in bone formation between donors, implants from individual donors were evaluated at the same cell dose. When bone generated by 2,000 hVSEL cells/implant were compared from donor 1 and 3, results showed these groups performed equally, but both generated more mineralized tissue than donor 2 (FIG. 6B). Yet donor 1 generated significantly more hVSEL cells than the other two donors. Total VSEL cells generated were 312K, 19K and 11K for donor 1, 2 and 3, respectively. Moreover, when fewer cells/implant were compared to the 2,000 hVSEL cell/implant of donor 2, more bone was formed, suggesting potential individual differences in hVSEL cell function.

Evaluation of Bone Formation by Histological Analysis.

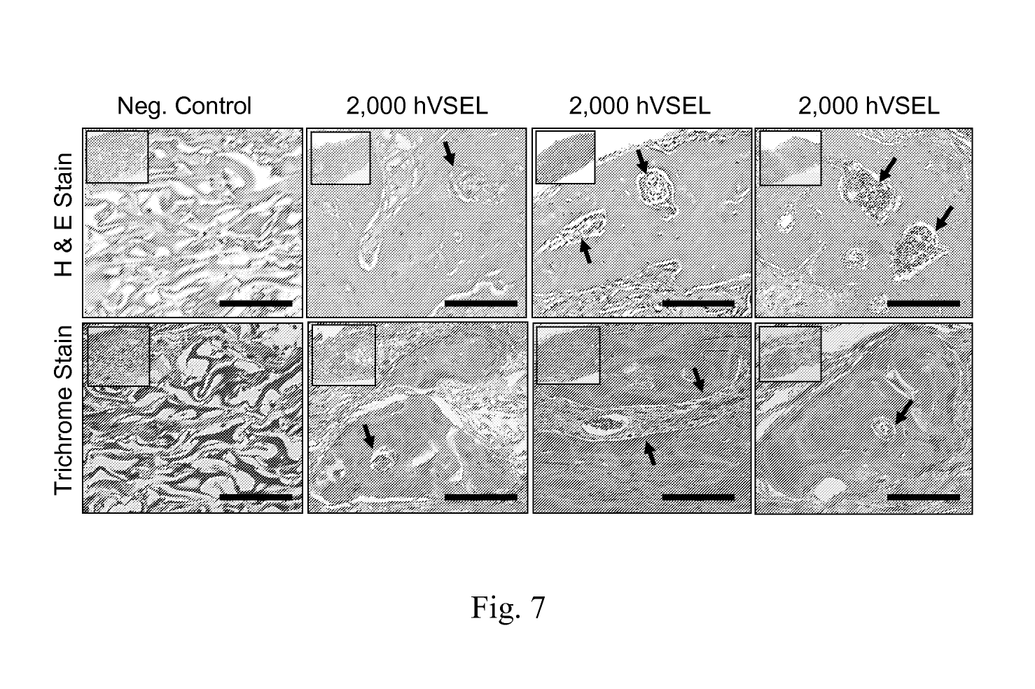

After decalcification, serial sections were generated through each defect in preparation for histologic evaluation. Implant material demonstrated a high degree of biocompatibility, characterized by very low levels of inflammation and no evidence of a foreign body response. In the control group, the collagen Gelfoam carrier matrix persisted and no osteoid or mature lamellar bone could be observed by H&E or Masson's trichrome staining (FIG. 7). In experimental groups implanted with 2,000-30,000 hVSEL cells, lamellar bone containing marrow spaces was observed within the calvarial defect (FIG. 7B-D). Here the carrier matrix was largely resorbed from the defect site with few remaining particles embedded within fibrous connective tissue surrounding the lamellar bone. The bone generated was comprised of predominantly of mature lamellar bone (FIG. 7B-D).

Demonstration that hVSEL Cells Formed Bone.

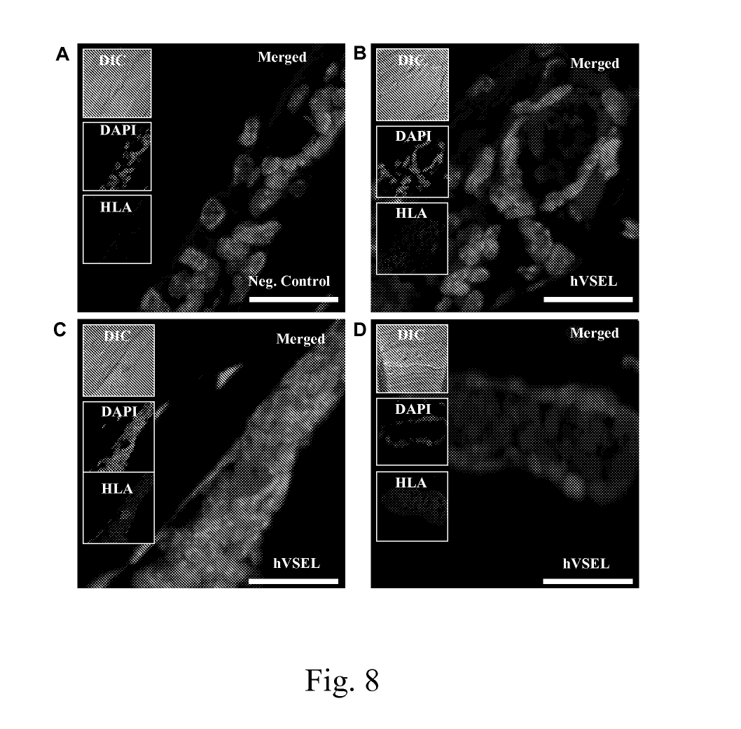

To validate that the bone formed was indeed generated by the implanted human cells, two independent methods were evaluated. First, the sections were stained with a pan-human specific leukocyte antigen (HLA) antibody. Mice implanted with vehicle only, containing no human VSELs did not demonstrate any cross-reactivity for human HLA (FIG. 8A). Mice implanted with human VSEL cells demonstrated significant human HLA staining specifically on osteoblasts and in osteocytes (FIG. 8B-D).

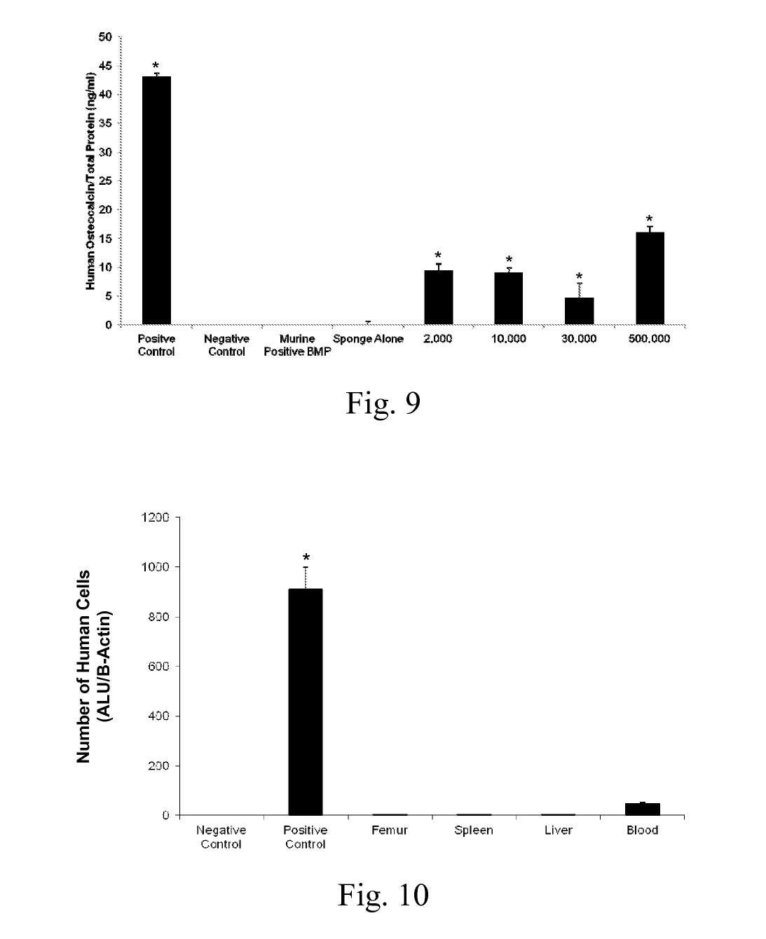

To further demonstrate that the hVSEL cells generated bone, serum from the animals collected at the time of sacrifice (3 months) was evaluated for the presence of human specific osteocalcin. Animals that did not receive any hVSEL cells in their implants did not demonstrate any human osteocalcin in the serum (FIG. 9). Animals which were implanted with hVSEL cells did have circulating human osteocalcin present within their serum. The levels of osteocalcin roughly corresponded to the amount of bone formation with animals receiving 500,000 cells/implant have the highest concentration of osteocalcin present in the serum, and animals implanted with fewer VSEL cells demonstrating less osteocalcin in the blood.

Localization of Human Cells to the Bone Defects.

Migration of human VSELs from the implant site into the periphery was assessed using quantitative real-time PCR for human specific Alu sequences. The presence of human DNA was evaluated from representative tissue samples from the spleen, femur, right lobe of the liver and whole blood. Animals not implanted with human cells and animals implanted with murine bone marrow served as a negative controls. A human prostate cancer cell line (PC-3) mixed (1:1,000) with murine bone marrow served as a positive control. Human specific Alu expression was identified at low levels present in the blood of the recipient animals which were implanted with human VSEL cells (FIG. 10). No evidence of human DNA was seen in the spleen, femur or liver of the animals implanted with human VSEL cells suggesting that the level of VSEL cells that migrated out of the defect was relatively low.

### Example 4

### Human VSELs can Regenerate Cartilage

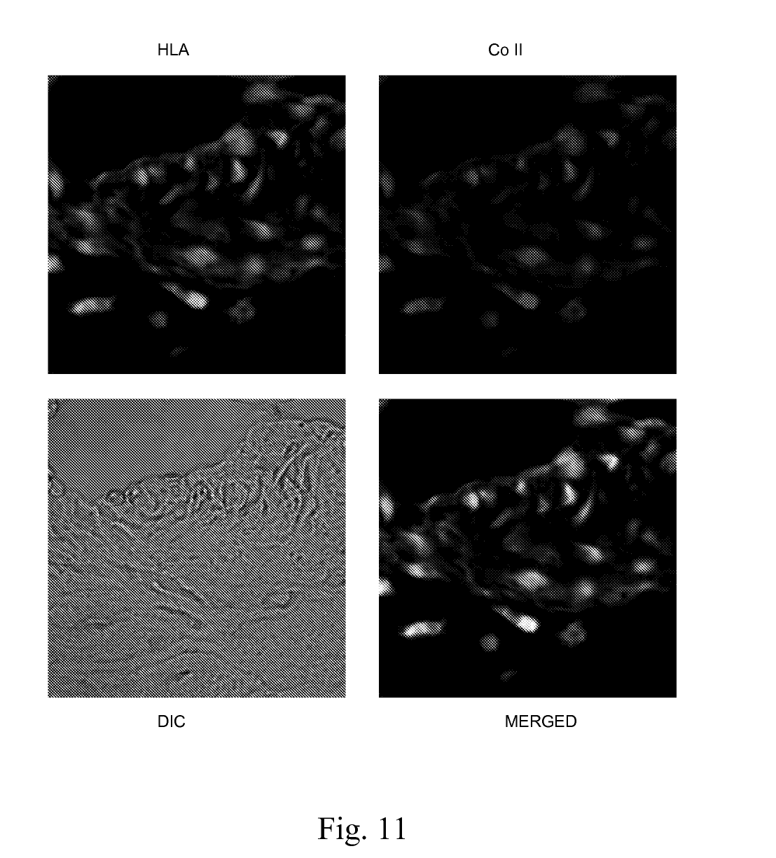

Localization of human cells to cartilage. Histological examination in several sections indicated the presence of human VSELs in cartilage. In several sections, woven bone was observed with tissues that were reminiscent of chondrocytes embedded in cartilage. Further analyses were performed to determine if this cell type produced collagen type II which would indicate cartilage formation. Tissues formed by hVSEL cells were immunostained with an fluorescent human specific pan-human leukocyte antigen (HLA) and stained for collagen type II (Co II). FIG. 11 shows merged HLA, CO II, and differential interference contrast (DIC) images.

While the present invention has been described with reference to the specific embodiments thereof it should be understood by those skilled in the art that various changes may be made and equivalents may be substituted without departing from the true spirit and scope of the invention. In addition, many modifications may be made to adapt a particular situation, material, composition of matter, process, process step or steps, to the present invention. All such modifications are intended to be within the scope of the claims appended hereto.

### REFERENCES

1. Rodgerson, D. O. and Harris, A. (2011) A Comparison of Stem Cells for Therapeutic Use. Stem Cell Rev and Rep. Pub. on line DOI 10.1007/s12015-011-9241
2. Taichman, R. S., Wang, Z., Shiozawa, Y., Jung, Y., Song, J., Balduino, A., Wang, J., Patel, L. R., Havens, A., Kucia, M., Ratajczak, M., Krebsbach, P. H. Prospective Identification and Skeletal Localization of Cells Capable of Multi-Lineage Differentiation In Vivo. Stem Cells Dev. 2010.
3. Kucia, M., Wu, W., Ratajczak, M. Z. Bone marrow-derived very small embryonic-like stem cells: Their developmental origin and biological significance. Dev. Dyn. 2007.
4. Kucia, M., Zuba-Surma, E. K., Wysoczynski, M., Wu, W., Ratajczak, J., Machalinski, B., Ratajczak, M. Z. Adult marrow-derived very small embryonic-like stem cells and tissue engineering. [Review] [104 refs]. Expert Opinion on Biological Therapy 7(10):1499-514, 2007.
5. Kucia, M., Reca, R., Campbell, F. R., Zuba-Sunna, E., Majka, M., Ratajczak, J., Ratajczak, M. Z. A population of very small embryonic-like (VSEL) CXCR4(+)SSEA-1(+)Oct-4+ stem cells identified in adult bone marrow. Leukemia 1920; 857-869.

We claim:

1. A method for treating damage or injury to osteochondral tissue in a subject, comprising administering to the subject an effective amount of a composition comprising very small embryonic like stem cells (VSELs), wherein the VSELs differentiate to treat the osteochondral tissue.

2. The method of claim 1, wherein the VSELs differentiate to osteoblasts.

3. The method of claim 1, wherein the VSELs differentiate to chondrocytes.

4. The method of claim 1, wherein the VSELs are administered directly to the tissue.

5. The method of claim 1, wherein the VSELs are administered systemically.

6. The method of claim 4, wherein the composition comprises about 20 to about 5×10⁵ VSELs per mm³.

7. The method of claim 4, wherein the composition comprises about 40 to about 4,000 VSELs per mm³.

8. The method of claim 4, wherein the composition comprises about 10 to about 1×10⁵ VSELs per mm².

9. The method of claim 4, wherein the composition comprises about 25 to about 500 VSELs per mm².

10. The method of claim 4, wherein the composition comprises a suitable matrix.

11. The method of claim 10, wherein the matrix is biodegradable.

12. The method of claim 1, wherein the composition comprises one or more bone morphogenic proteins (BMPs).

13. The method of claim 1, wherein the osteochondral tissue is bone.

14. The method of claim 1, wherein the osteochondral tissue is cartilage.

15. The method of claim 1, wherein the osteochondral tissue is articular cartilage.

16. The method of claim 1, wherein the composition comprises cells that are at least about 50% VSELs.

17. The method of claim 1, wherein the composition comprises cells that are at least about 70% VSELs.

18. The method of claim 1, wherein the composition comprises cells that are at least about 90% VSELs.

19. The method of claim 1, wherein the VSELs are autologous VSELs.

20. The method of claim 1, wherein the VSELs are allogeneic VSELs.

21. The method of claim 1, wherein the VSELs are human.

22. The method of claim 1, wherein the method is used to treat osteogenesis imperfecta.

23. The method of claim 1, wherein the method is used to treat osteoarthritis.

24. The method of claim 1, wherein the method is used to treat osteoporosis.

25. An stem cell composition comprising an effective amount of VSELs sufficient for regeneration or repair of an osteochondral tissue.

26. The stem cell composition of claim 25, which comprises about 20 to about 5×10⁵ VSELs per mm³.

27. The stem cell composition of claim 25, which comprises about 40 to about 4,000 VSELs per mm³.

28. The stem cell composition of claim 25, which comprises about 10 to about 1×10⁵ VSELs per mm².

29. The stem cell composition of claim 25, which comprises about 25 to about 500 VSELs per mm².

30. The stem cell composition of claim 25, which comprises a suitable matrix.

31. The stem cell composition of claim 30, wherein the matrix is biodegradable.

32. The stem cell composition of claim 25, which comprises one or more bone morphogenic proteins (BMPs).

33. The stem cell composition of claim 25, which is differentiable into osteoblasts.

34. The stem cell composition of claim 25, which is differentiable into chondrocytes.

35. The stem cell composition of claim 25, which comprises cells that are at least about 50% VSELs.

36. The stem cell composition of claim 25, which comprises cells that are at least about 70% VSELs.

37. The stem cell composition of claim 25, which comprises cells that are at least about 90% VSELs.


SEQUENCE LISTING (3 sequences)

SEQ ID NO: 1 - DNA, 21 bp, Artificial Sequence
  synthetic primer
catggtgaaaccccgtctcta

SEQ ID NO: 2 - DNA, 19 bp, Artificial Sequence
  synthetic primer
gcctcagcctcccgagtag

SEQ ID NO: 3 - DNA, 20 bp, Artificial Sequence
  probe
attagccgggcgtggtggcg